(19) United States
(12) Patent Application Publication
(10) Pub. No.: US 2007/0048865 A1
(43) Pub. Date: Mar. 1, 2007

(54) Method for acceleration of stem cell differentiation

(76) Inventors: Kenzaburo Tani, Fukuoka-city (JP); Ryo Kurita, Fukuoka-city (JP)

Correspondence Address: HELLER EHRMAN LLP, 275 MIDDLEFIELD ROAD, MENLO PARK, CA, 94025-3506, US

(21) Appl. No.: 11/377,847

(22) Filed: Mar. 15, 2006

Related U.S. Application Data
(60) Provisional application No. 60/662,286, filed on Mar. 15, 2005.

(51) Int. Cl. C12N 15/86; C12N 5/06; C12N 5/08

(52) U.S. Cl. 435/456; 435/362; 435/366

(57) ABSTRACT

The present invention relates to methods for accelerating the differentiation of embryonic stem cells by introducing nucleic acid encoding the Tal1/Scl transcription factor into the cells, and culturing the transduced cells in a differentiation medium.

### CROSS-REFERENCE TO RELATED APPLICATIONS

This is a non-provisional application 37 C.F.R. §1.53(b), claiming priority under 37 C.F.R. §119(e) to U.S. Provisional Patent Application Ser. No. 60/662,286 filed on Mar. 15, 2005, the entire disclosure of which is hereby expressly incorporated by reference.

### BACKGROUND OF THE INVENTION

1. Field of the Invention

The present invention concerns a method for stem cell differentiation. In particular, the invention concerns a method for accelerating the differentiation of stem cells, such as embryonic stem cells, by introducing nucleic acid encoding the Tal1/Scl transcription factor into the cells, and culturing the transduced cells in a differentiation medium.

2. Related Art

Embryonic stem cells (ES cells) are derived from totipotent cells from early mammalian embryo and are capable of unlimited undifferentiated proliferation in vitro (Evans and Kaufman, Nature, 292:154 (1981); Martin, G., Proc. Natl. Acad. Sci. USA, 78:7634 (1981). ES cells can differentiate into any cell type in vivo (Evans et al., Nature, 292:154-156 (1981); Bradley, et al., Nature, 309:255-256 (1984)), and into a variety of cells in vitro (Doetschman, et al., J. Embryol. Exp. Morph., 87:27-45 (1985); Wobus, et al., Biomed. Biochim. Acta, 47:965-973 (1988); Robbins, et al., J. Biol. Chem., 265:11905-11909 (1990)). Non-human primate and human embryonic stem cell lines have been established (see, e.g., Thomson and Marshall, Curr Top Dev Biol, 38:133-65 (1998); Thomson et al., Proc Natl Acad Sci USA, 92:7844-7848 (1995); and Thomson et al., Science, 282:1145-1147 (1998)). Primate embryonic stem cells are described, for example, in U.S. Pat. No. 5,843,780 issued on Dec. 1, 1998. Monkey-origin embryonic stem cells are disclosed in U.S. Patent Application Publication No. 2003/0157710 A1, published on Aug. 21, 2003.

Adult stem cells (also referred to as tissue stem cells, somatic stem cells or post-natal stem cells) have also been isolated from numerous adult tissues, umbilical cord, and other non-embryonic sources, and shown to be able to differentiate into other tissue and cell types.

Various culture conditions have been evaluated for inducing in vitro differentiation of stem cells, in particular ES cells into various cell types, such as, for example, cardiomyocytes, hematopoietic progenitors, yolk sac, skeletal myocytes, smooth muscle cells, adipocytes, chondrocytes, endothelial cells, melanocytes, neurons, glia, pancreatic islet cells, and primitive endoderm. Vascular endothelial growth factor (VEGF), basic fibroblast growth factor (bFGF) and morphogenetic protein 4 (BMP4) have been reported to promote primitive or definitive hematopoietic development in murine ES cells (Johansson and Wiles, Mol. Cell. Biol., 15:141-151 (1995); Faloon et al., Development, 127:1931-1941 (2000); and Nakayama et al., Blood, 95:2275-2283 (2000)). Hematopoietic differentiation from non-human primate ES cells in the presence of cytokines and/or growth factors, such as granulocyte colony-stimulating factor (GCSF), erythropoietin (EPO), interleukin-3 (IL-3), stem cell factor (SCF), thrombopoietin (TPO), vascular endothelial growth factor (VEGF), and bone morphogenetic protein (BMP) has been has been tested by Umeda et al., Development, 131:1869-1879 (2004). Especially exogenous VEGF, bFGF and EPO proved to be effective in this study.

Since the successful establishment of human embryonic stem (ES) cell lines in 1998, (Thomson et al., Science, 282:1145-1147 (1998)) transplantation of differentiated embryonic stem cells to specific organs have been expected to have great therapeutic potentials.

### SUMMARY OF THE INVENTION

The present invention is based, at least in part, on experimental data demonstrating that introduction of nucleic acid encoding the basic helix-loop-helix transcription factor Tal1/Scl (T cell acute lymphoblastic leukemia/stem cell leukemia transcription factor) into ES cells facilitates the differentiation of ES cells into hematopoietic cells.

In one aspect, the invention concerns a method for accelerating differentiation of embryonic stem cells, comprising introducing nucleic acid encoding the Tal1/Scl transcription factor into embryoid bodies (EBs) formed from undifferentiated embryonic stem cells, and culturing the EBs carrying the TL1/SCl encoding nucleic acid in a differentiation medium until differentiation is confirmed.

In one embodiment, the differentiation is hematopoietic differentiation.

In another embodiment, the nucleic acid is introduced into the EBs by vector-mediated gene transfer.

In yet another embodiment, the vector is a viral vector, in particular a retroviral vector, preferably a lentiviral vector, such as, for example, a VSV-G pseudotyped lentiviral vector. The lentiviral vectors may carry the Tal1/Scl gene under control of a variety of promoters known in the art, including, without limitation, EF-1α, CAG, PGK and CMV promoters.

Hematopoietic differentiation can be confirmed by methods known in the art, such as by confirming the presence of multilineage blood cells, an immunochemical test, morphological analysis, by detecting regional expression of at least one embryonic marker specific for a cellular lineage, or any combination of such detection methods.

In a particular embodiment, the embryonic marker is selected from the group consisting of zeta-globin, neurofilament 68 kD, and alpha-fetoprotein. Differentiation of ES cells can be performed following any technique, in any medium known in the art, where the medium typically contains one or more cytokines and/or growth factors, such as, for example, IL-3, IL-6, GM-CSF, G-CSF, SCF, and/or erythropoietin (EPO).

### BRIEF DESCRIPTION OF THE DRAWINGS

FIG. 1-1 shows hematopoietic cells obtained from Tal1/Scl gene tranduced marmoset EB cells.

FIG. 1-2 shows the identification of erythroid series cells derived from Tal1/Scl gene transduced marmoset EB cells using anti-human hemoglobin antibody.

FIG. 1-3 shows the identification of granulocyte-macrophage series cells derived from Tal1/Scl gene transduced marmoset EB cells using double esterase staining method.

### DETAILED DESCRIPTION OF THE PREFERRED EMBODIMENT

A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provides one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

The term “embryonic stem cell” is used herein to refer to primitive (undifferentiated) cells from the embryo that have the potential to differentiate into a wide variety of specialized cell types.

The term “adult stem cell” is used herein to refer to an undifferentiated cell found in a differentiated tissue, that can renew itself and (with certain limitations) differentiate into all specialized cell types of the tissue from which it originated.

The term “hematopoietic stem cell” is used to refer to a stem cell from which all red and white blood cells evolve.

Detailed Description

The practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2^{nd} edition (Sambrook et al., 1989); “Animal Cell Culture” (R. I. Freshney, ed., 1987); “Gene Transfer Vectors for Mammalian Cells” (J. M. Miller & M. P. Calos, eds., 1987); “Current Protocols in Molecular Biology” (F.M. Ausubel et al., eds., 1987); and “Embryonic Stem Cells: Methods and Protocols” (Kursad Turksen, ed., Humana Press, Totowa N.J., 2001.

The present invention provides improved methods for stem cell differentiation, in particular for the differentiation of ES cells. Protocols for maintaining and differentiating stem cells in cell culture are known in the art. Such techniques include, without limitation, protocols for the isolation of ES cells, preparation of media, ES cell passaging techniques, ES cell cryopreservation techniques, ES cell transfection techniques, techniques for analysis of ES cells and their differentiated derivatives, and techniques for ES cell differentiation into various cells types, including, for example, hematopoietic, neural, or cardiac cells.

Human and non-human primate ES cells are typically isolated by transferring the inner cell mass into a culture medium. Traditionally, the suface of the cell culture dish is coated with mouse embryonic skin cells, treated to inhibit their differentiation (usually referred to as a “feeder layer”). The feeder layer releases nutrients into the cell culture. It is, however, also possible to culture ES cells in the absence of such feeder layer. After allowing time for proliferation, the ES cells are passaged multiple times to yield pluripotent ES cell lines, which can be frozen and maintained without differentiation.

Differentiation of ES cells into different cell types can be induced and controlled in various ways, such as, for example, by changing the composition of the cell culture medium, culturing on certain stromal cells, or modifying the ES cells by insertion of various genes. Basic protocols for directed differentiation of ES cells, including differentiation media, are provided, for example, in Chapters 5-9 and Appendices B and C of the NIH report: Stem Cells: Scientific Progress and Future Research Directions, June 2001, http://stemcells.nih.gov/info/scireport, the entire content of which is hereby expressly incorporated by reference.

In brief, ESs cells, including primate, such as human, ES cells, begin to differentiate after being removed from feeder layers and allowed to grow in suspension culture on a non-adherent surface. As a result, embryoid bodies (EBs) are formed, which can be dissociated and replated in monolayer cultures and exposed to specific growth factors that influence further differentiation.

Some growth factors induce cell types that would normally be derived from ectoderm in the embryo; such as, for example, retinoic acid, epidermal growth factor (EGF), bone morphogenic protein 4 (BMP4), and basic fibroblast growth factor (bFGF). Other growth factors, such as activin-A and transforming growth factor-beta 1 (TGF-β1) trigger the differentiation of mesodermally derived cells. Hepatocyte growth factor (HGF) and nerve growth factor (NGF) promote differentiation into all three germ layers, including endoderm. The identity of the differentiated human EB-derived cells can be determined by their morphology, growth characteristics and expression of messenger RNA (mRNA) for specific markers (see, e.g., Shamblott et al., Proc. Natl. Acad. Sci. USA, 95:13726-13731 (2001).

The differentiation of human ES cells into hematopoietic precursor cells can, for example, be achieved by co-culturing human ES cells with mouse bone marrow stromal cells (irradiated to prevent their replication) in growth medium that contains fetal bovine serum, but no added growth factors. The partly differentiated cells express CD34, a marker for blood cell precursors. If these partly differentiated human ES cells are replated under conditions that allow them to form colonies of hematopoietic cells, they differentiate into erythroid cells, macrophages, granulocytes, and megakaryocytes (Odorico et al., Stem Cells, 19:193-204 (2001)).

Media for differentiation of human ES cells into hematopoietic colony-forming cells are disclosed in several publications, such as, for example, Kaufman et al., Proc. Natl. Acad. Sci. USA, 98:10716-10721 (2001). A particular medium for differentiation of primate ES cells into hematopoietic colony-forming cells is described in the Example below.

Cell markers suitable for identification of primate, including human, hematopoietic stem cells include CD34⁺, CD59⁺, Thy1⁺, CD38^{low/-}, C-kit^{-/low}, lin⁻. These markers can be used to confirm hematopoietic differentiation, for example by using antibody-based detection techniques.

According to the present invention, ES cell differentiation is accelerated by transducing a viral, preferably lentiviral, vector carrying the Tal1/Scl gene into ES cells, in particular embryoid bodies (EBs) formed from ES cells.

Vectors derived from lentiviruses are well known in the art and are considered an efficient gene delivery system. Lentiviral vectors derived from human immunodeficiency virus (HIV) (Kafri et al., Nat. Genet., 17:314-317 (1997); Naldini et al., Science, 272:163-167 (1996); Peng et al., Gene Ther., 8:1456-1463 (2001)), feline immunodeficiency virus (FIV) (Johnston et al., J. Viol., 73:4991-5000 (1999); Poeschla et al., Nat. Med., 4:354-357 (1998); Wang et al., Am. J. Respir. Cell. Mol. Biol., 22:129-138 (1999)), and others have been utilized in vitro and in vivo to transfer genes to somatic cells. One of the attractions of lentivirus-based vectors is that they can transduce both dividing and nondividing cells, resulting in stable integration and long-term expression of the transgene. For the purpose of the present invention, lentiviral vectors pseudotyped with the vesicular stomatitis virus G (VSV-G) are particularly useful, but other lentiviral vectors are also included.

Further details of the invention will be apparent from the following non-limiting Example.

### EXAMPLE

### Acceleration of Stem Cell Differentiation

ES cells are pluripotent cells which are expected to become one of the most important transplantable cell sources for future medicine. However, to estimate the safety and efficacy of ES cell therapy in vivo, the preclinical studies using animal models, including non-human primates, are essential. It has already been demonstrated that non-human primates, such as common marmosets (CM), are suitable as laboratory animal models. The present example focuses on the induction of hematopoietic stem cells in this model system, using gene transduction.

To introduce exogenous DNA into CMES, VSV-G pseudotyped human immunodeficiency virus (lentiviral) vectors containing EF1a promoter and several kinds of hematopoietic genes such as tal1/scl, gata1, gata2, lh2, and hoxB4 genes were constructed and introduced into CM ES cells using the following protocol.

Common marmoset ES cells were harvested from irradiated (40Gy) embryonic fibroblast cell layers using 0.25% trypsin/1 mM EDTA and the 1-2×104 cells were replated to 9 cm diameter dish to obtain embryoid bodies (EB) in IMDM containing 15% fetal bovine serum, 200 μg/ml of transferring, 50 μg/ml ascorbic acid, 10 μg/ml insulin, 0.45 mM monothioglycol, 100 μg/ml streptomycin and 100 U/ml penicillin. The EBs obtained were transduced with VSV-G pseudotype lentiviral vector (Bai et aL, Gene Ther., 10:1446-1457 (2003)) which can express cDNA encoding the listed helatopoietic genes, including tal1/scl cDNA, under the promoter of EF-1 alpha for 1-2 days.

The cDNA transduced cells were cultured for further 10 to 14 days, in the absence of cytokines. The medium was changed to fresh one every 3-4 days. Subsequently, for CFU (colony forming unit) assays, the cells were plated to semisolid medium consisting of IMDM containing 1% methylcellulose, 30% fetal bovine serum, 1% bovine serum albumin, 3 U/ml erythropoietin, 10⁻⁴ M 2-mercaptoethanol, 2 mM L-glutamine, 50 ng/ml SCF (stem cell factor), 20 ng/ml GM-CSF (granulocyte macrophage colony stimulating factor), 20 ng/ml IL (interleukin)-3, 20 ng/ml IL-6, 20 ng/ml G-CSF (granulocyte colony stimulating factor) (StemCell Technologies). The cells obtained from each colony were harvested, washed with IMDM medium and cytospinned to glass slides. The cells were stained with MayGiemsa staining solution, esterase staining solution and antibodies following standard methods (Umeda et al., Development, 131(8):1869079 (2004)).

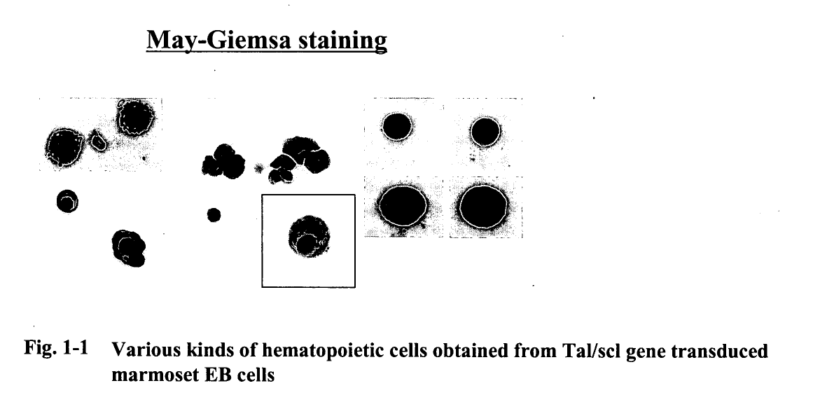

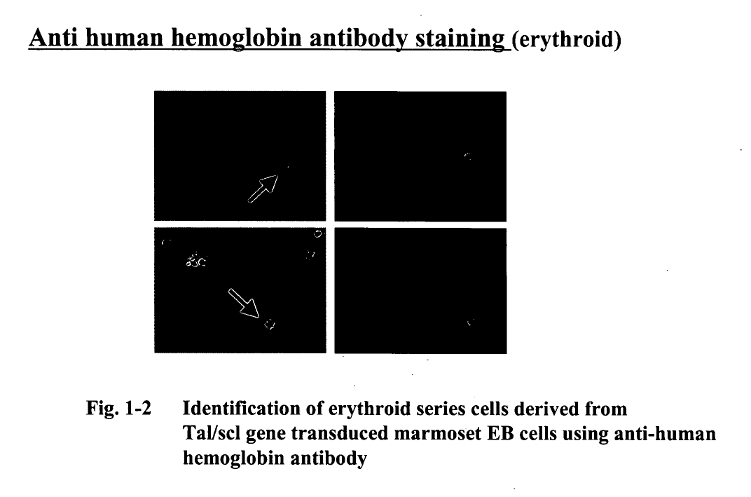

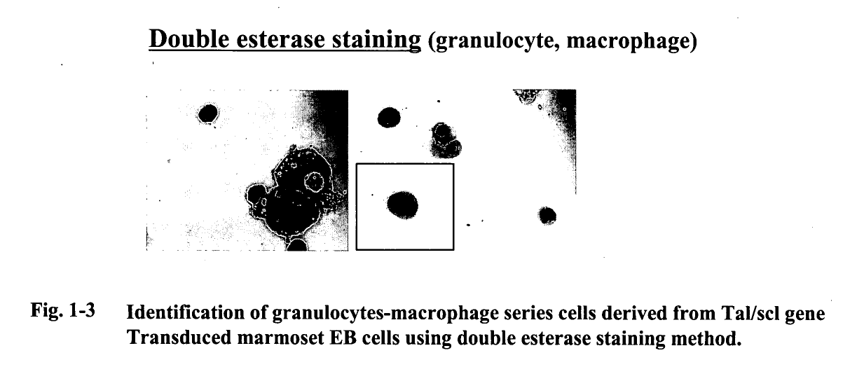

In the absence of cDNA transduction, no obvious CFU cells were observed. Very occasionally, a few CFU-M (colony forming units of monocytes) were observed in each dish. CFU's were observed in cDNA transduced cells, however, only when the HIV vector containing tal1/scl cDNA was introduced, was hematopoietic induction from CM ES cells dramatically increased. Thus, a significant number of CFUs were observed in each dish where the tal/scl transduced EB cells were plated. More than 40 CFU colonies (CFU: 20-30%, CFU-GM: 5-10%, CFU-M: 60-75%) were observed from 10⁵ EB cells. These colonies were microscopically identified to contain erythroid, myeloid, monocyte-macrophage, and megakaryoid cells after the cytospinned cells were stained with May-Giemsa, double esterase and antihemoglobin antibody (FIGS. 1-1-1-3). The data were reproducible for three times. The results demonstrate that the tal1/scl cDNA transduction efficiently accelerates the hematopoietic differentiation of marmoset ES cells and is expected to be broadly useful to differentiate other primate, including human, ES cells. ES-tal1/scl cells can be xenotransplanted into the immunodeficient mice to confirm their long-term hematopoietic reconstitution capacity and the safety of the tal1/scl transduced ES cells.

All references cited throughout this disclosure are hereby expressly incorporated by reference in their entirety.

What is claimed is:

1. A method for accelerating differentiation of embryonic stem cells, comprising introducing nucleic acid encoding the Tal1/Scl transcription factor into embryoid bodies formed from undifferentiated embryonic stem cells, and culturing the embryoid bodies carrying the Tal1/Scl encoding nucleic acid in a differentiation medium until differentiation is confirmed.

2. The method of claim 1 wherein said differentiation is hematopoietic differentiation.

3. The method of claim 2 wherein said nucleic acid is introduced into said embryoid bodies by vector-mediated gene transfer.

4. The method of clam 3 wherein said vector is a viral vector.

5. The method of claim 4 wherein said vector is a lentiviral vector.

6. The method of claim 5 wherein said lentiviral vector is VSV-G pseudotyped.

7. The method of claim 6 wherein said lentiviral vector comprises the Tal1/Scl gene under control of a promoter selected from the group consisting of EF-1a, CAG, PGK and CMV promoters.

8. The method of claim 7 wherein said promoter is an EF-1a promoter.

9. The method of claim 2 wherein differentiation is confirmed by confirming the presence of multilineage blood cells.

10. The method of claim 9 wherein the presence of multilineage blood cells is confirmed by an immunochemical test.

11. The method of claim 9 wherein the presence of multilineage blood cells is confirmed by morphological analysis.

12. The method of claim 2 wherein differentiation is confirmed by detecting regional expression of at least one embryonic marker specific for a cellular lineage.

13. The method of claim 12 wherein said embryonic marker is selected from the group consisting of zeta-globin, neurofilament 68 kD, and alpha-fetoprotein.

14. The method of claim 2 wherein said differentiation medium comprises one or more cytokines and/or growth factors.

15. The method of claim 2 wherein said differentiation medium comprises one or more ingredients selected from the group consisting of IL-3, IL-6, GM-CSF, G-CSF, SCF, and erythropoietin.

16. The method of claim 15 wherein said differentiation medium comprises all of said ingredients.

17. The method of claim 1 wherein said embryonic stem cell is a primate cell.

18. The method of claim 17 wherein said primate is a non-human primate.

19. The method of claim 18 wherein said non-human primate is a marmoset.

20. The method of claim 17 wherein said primate is a human.
